(19) United States
(12) Patent Application Publication
(10) Pub. No.: US 2004/0132064 A1
(43) Pub. Date: Jul. 8, 2004

(54) Ratios of collagen peptides, their uses and products

(75) Inventor: A. Robin Poole, South Lancaster (CA)

Correspondence Address: STEPHEN B MAEBIUS, FOLEY AND LARDNER, 3000 K STREET N W SUITE 500, WASHINGTON, DC, 20007-5109, US

(73) Assignee: Shriners Hospital For Children

(21) Appl. No.: 10/673,647

(22) Filed: Sep. 30, 2003

Related U.S. Application Data
(60) Provisional application No. 60/414,324, filed on Sep. 30, 2002.

(51) Int. Cl. C12Q 1/68

(52) U.S. Cl. 435/6

(57) ABSTRACT

The present invention provides a ratio of two collagen-derived peptides which can be used to track the progression of joint destruction in osteoarthritis and rheumatoid arthritis or any destructive arthritis. In addition, the ratio can be used to screen for agents to treat osteoarthritis and rheumatoid arthritis. Furthermore, it can also be used to monitor efficacy of current therapeutic regimens and determine the type of therapy that should be used to treat the arthritis (aggressive versus non-aggressive).

### CROSS REFERENCE TO RELATED APPLICATIONS

This application is based on U.S. provisional patent application serial No. 60/141,324, filed Sep. 30, 2002. The entire contents of this application, including its specification, claims and drawings, are incorporated herein by reference in their entirety.

### FIELD OF THE INVENTION

The present invention relates to ratios of collagen peptide epitopes recognized by antibodies, their use in treatment, diagnosis, detection and monitoring of disease activity and progression and conditions relating to collagen, assay kits to determine the ratios, screening methods employing the ratios to determine the ability of agents to affect the ratios, and agents identified by such screening methods.

### BACKGROUND OF THE INVENTION

Osteoarthritis (OA), a debilitative and painful condition, represents a complex of interactive degradative and reparative degenerative processes in cartilage and bone with secondary inflammatory changes. It results in a progressive degeneration of diarthrodial joints in particular a loss of articular cartilage, resulting in a loss of joint function. Recent studies have demonstrated that excessive degradation, involving cleavage and denaturation of most particularly (but not exclusively) type II collagen in human articular cartilage is implicated in osteoarthritis (Hollander, A. P. et al., J. Clin. Invest. 93:1722-1732 (1994); Dodge, G. R. and Poole, A. R., J. Clin. Invest. 83:647-61 (1989); Hollander, A. P. et al., J. Clin. Invest. 96:2859-69 (1995); Billinghurst, R. C. et al., J. Clin. Invest. 99:1534-45 (1997); Dahlberg, L. et al., Arthritis Rheum. 43:673-82 (2000); Wu, W. et al., Arthritis Rheum. 46:2087-2094 (2002).

Primary or idiopathic OA affects interphalangeal joints, and other small joints as well as large joints, such as the hip or knee. Involvement of the proximal interphalangeal joints of the hands leads to the formation of Bouchard's nodes in contrast, involvement of the distal interphalangeal joints involves Heberden's nodes. The disease may involve one particular joint, or it may be more generalized and involve multiple joints. OA may be genetically transmitted (such as a consequence, for example, of a mutation in the type II collagen COL2A1 gene) and therefore is known as familial OA. OA may develop in patients after traumatic injury or damage to chondrocytes associated with abnormal deposits of the cartilage matrix found in metabolic diseases such as hemochromatosis, ochronosis or alkaptonuria, Wilson's disease, and Gaucher's disease. Idiopathic OA may result from disturbances in cartilage metabolism caused by endocrine disorders (Poole A. R., and Howell D. S., Etiopathogenesis of Osteoarthritis. In: Diagnosis and Medical/Surgical Management (3^{rd} ed.), Moskowitz, R. W. et al. (Eds), pp. 29-37, Philadelphia, Saunders Company (2001). Mineralization of cartilage matrix is a feature of OA and is associated with chondrocyte hypertrophy. See Poole, A. R. and Howell, D. S. (2001) supra.

Rheumatoid arthritis is another example of a disease of the musculoskeletal system in which joint cartilages are destroyed as part of an inflammatory condition involving inflamed synovium lining the joint cavity. See Henderson, B., Edwards, J. C. W., Pettipher, E. R. (eds), Mechanisms and Models in Rheumatoid Arthritis, London, Academic Press, 1995. This inflammation involves the erosive destruction of articular cartilage and its type II collagen by collagenases as well as destruction of bone containing type I collagen (see Henderson, B. et al. supra). Rheumatoid arthritis usually involves multiple joints.

Assays of collagen epitopes have been used to detect the resorption of bone such as in osteoporosis (type I collagen cross-links) (see Delmas, P. and Garnero, P. Biological markers of bone turnover in osteoporosis. In J. C. Stevenson, R E. Lindsay, (eds), Osteoporosis, pp. 117-136, London, Chapman and Hall, 1998) and the turnover/resorption of hyaline cartilage in arthritis (type II collagen degradation products) (see Garnero P. et al., Arthritis Rheumatis 43:953-961, 2000 [Robin Poole to check]).

At the present time, joint space narrowing, as determined radiographically by x-ray analysis, is ordinarily used to determine loss of articular cartilage in osteoarthritis or rheumatoid arthritis or in any other types of arthritis. This may require hundreds of patients and a two-three year period of study to accurately measure in population studies, such as in clinical trials for drug efficacy, loss at joint space with disease progression in osteoarthritis. Due to the more rapid erosive character of rheumatoid arthritis (RA), a period of 1-1½ years may be sufficient to determine joint space loss in RA. Even so the expense of such studies, as in clinical trials to assess potential disease modifying drugs, is extremely costly in view of the long periods of time and large numbers of patients required for such studies. It would, therefore, be of great value to measure not only disease progression (as measured by joint space narrowing) but disease activity/process at a specific time point which is reflective and predictive of outcome. This may be possible by the use of biological markers such as those for cartilage collagen degradation in combination as well as singly. Such information would also be of value in deciding upon the type of therapy that may be administered to the patient. For example in rheumatoid arthritis, identification of rapid progressors would favour the use of a more aggressive therapy to control the more rapid joint destruction.

Therefore, there is a need to detect disease activity and predict disease progression. There is also a need to have a means, such as immunoassays of cartilage collagen degradation products in blood or joint fluid, to more rapidly determine the efficacy of joint agents that may prevent cartilage destruction. Accordingly, there is a obviously a need to develop a single assay and combination of assays to provide a ratios of collagen epitopes in the detection of the relative cleavage and synthesis of type II collagen in patients with osteoarthritis, rheumatoid arthritis, and other types of arthritis involving joint destruction.

### SUMMARY OF THE INVENTION

In accordance with one aspect of the invention, there is provided a method of monitoring a collagen-related disease (preferably osteoarthritis, rheumatoid arthritis, and other types of arthritis involving joint destruction) comprising determining a ratio of C1, 2C neoepitope to C2C neoepitope in a subject. A higher result is predictive of greater progression of osteoarthritis and a lower result is predictive of greater progression of rheumatoid arthritis. Similarly, an increase in the ratio indicates or relates to progression of osteoarthritis and a decrease in the ratio indicates a progression of rheumatoid arthritis. In another aspect of the invention, the subject does not exhibit generalized osteoarthritis or exhibits rheumatoid or other inflammatory erosive arthritis.

In accordance with yet another embodiment, the invention provides a method of monitoring arthritis involving joint destruction other than osteoarthritis, comprising determining a level of C1, 2C neoepitope to C2C neoepitope in a subject, wherein a change in the level indicates a change in rate of progression of joint destruction of the arthritis. There is also provided a method wherein a decrease in the level of C1, 2C neoepitope to C2C neoepitope in a subject is indicative of rheumatoid arthritis. A further embodiment of the invention includes a method of monitoring efficacy of a therapeutic regimen for treating a collagen-related disease (preferably osteoarthritis or rheumatoid arthritis), comprising determining a ratio of C1,2C epitope to C 2C epitope in a subject, a change in the ratio indicating a change in severity of the disease. Similarly, the subject herein does not exhibit generalized osteoarthritis.

Another embodiment is a method of identifying an agent for treating a collagen-related disease (preferably osteoarthritis or rheumatoid arthritis), comprising administering to a subject an agent to be tested, and determining a ratio of C1,2C epitope to C 2C epitope in the subject, a change in the ratio indicating a change in severity of the disease

A further embodiment is a pharmaceutical composition for a collagen-related disease (preferably osteoarthritis or rheumatoid arthritis) comprising an agent identified by the above method in an amount effective to change the ratio or reduce the change in the ratio relative to an untreated subject.

Yet another embodiment is a kit for determining a ratio of C1,2C neoepitope to C 2C epitope in a biological sample, said kit comprising:

(a) a monoclonal antibody which binds to said C2C epitope having the following first peptide sequence

C-G-G-E-G-P-P(OH)-G-P-Q-G (COL2-¾C_{long mono} peptide);

(b) a polyclonal or monoclonal antibody which binds to said C1, 2C neoepitope having the following second peptide sequence

C-G-P-P(OH)-G-P-Q-G (COL2-¾Cₛₕₒᵣₜ peptide);

(c) two solid supports for binding each of said first and second peptides;

(d) a first labelled antibody conjugated to a first enzyme to measure the binding of said monoclonal antibody to said first peptide containing the C2C neoepitope; and

(e) a second labelled antibody conjugated to a second enzyme to measure the binding of said polyclonal or monoclonal antibody to said second peptide containing the C1, 2C neoepitope.

Still another embodiment is an improvement in a method of treating a collagen-related disease (preferably osteoarthritis or rheumatoid arthritis), the improvement comprising determining a ratio of C1,2C neoepitope to C 2C neoepitope in a subject being treated, wherein a change in the ratio correlates to a change in progression of the disease.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples of infections where it obviously will be useful to those skilled in the prior art.

### DETAILED DESCRIPTION OF THE INVENTION

Identification of biomarkers of cartilage metabolism that predict osteoarthritis disease activity and/or progression should aid development of disease-modifying agents. The predictive value of serum markers for knee osteoarthritis progression may be different in those with versus those without generalized osteoarthritis.

Two serum biomarkers have been developed for the measurement of the cleavage by collagenase of cartilage type II collagen: the C2C epitope, C-G-G-E-G-P-P(OH)-G-P-Q-G (COL2-¾C_{long mono} peptide), specific for type II collagen (see U.S. Pat. No. 6,132,976, filed Jan. 22, 1998, the entire contents of which are incorporated herein by reference in their entirety) and the C1, 2C epitope, C-G-P-P(OH)-G-P-Q-G (COL2-¾Cₛₕₒᵣₜ peptide, which measures mainly type II but also type I collagen cleavage (see Billinghurst, R. C. et al., J. Clin. Invest. 99:1535-1545 (1997)). They can be used together or as separate assays to detect type II collagen degradation Samples may be removed with a syringe from peripheral blood and allowed to clot to produce serum for analysis or prevented from clotting, to permit analysis of plasma, by an anti-coagulant such as heparin or ethylenediamine tetraacetic acid.

Accordingly, the present invention relates to the determination of ratios of collagen epitopes in the detection of the relative cleavage and synthesis of type II collagen in patients with osteoarthritis, rheumatoid arthritis, and other types of arthritis involving joint destruction.

Unless otherwise specified, “a” or “an” means “one or more”.

The term “C2C” refers to “C”ollagen type “2” “C”leavage and is used in an assay specific for type II collagen.

The term “C1, 2C” refers to “C”ollagen type “1” & “2” “C”leavage and is used in an assay specific for both type I & II collagen.

The term “Col II” stands for type II collagen.

The term “¾” refers to the cleavage of type II collagen into two pieces, namely, (1) ¼ the length of the collagen, and (2) ¾ the length of the collagen. “C” refers to the “C-terminus” at the end of the ¾ collagen piece. The terms “short” and “long” refer to the neoepitope that is detected. Both assay kits described above detect the neoepitope that is revealed after collagen is cleaved. The “short” detects a shorter portion of the “Long” sequence of amino acids.

An antibody, as described herein, refers to an immunoglobulin molecule or a binding fragment thereof (either enzymatically or recombinantly produced).

An antibody fragment is a portion of an antibody such as F(ab′)₂, F(ab)₂, Fab′, Fab, Fv, sFv and the like. Regardless of structure, an antibody fragment binds with the same antigen that is recognized by the intact antibody. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

Neoepitopes are epitopes that are not expressed in the native protein (collagen) and are only exposed in protein that has been modified by a change in structure. Such a change in structure to reveal a neoepitope may follow proteolytic cleavage (collagenase), a conformational change following activation or following binding of the protein to another protein.

Osteophytes (Heberden's and Bouchard's nodes) refer to extra bone the body produces and deposits in an osteoarthritic joint that can impede its movement. These bony growths are also known as bone spurs. The osteophytes may be found in arthritic affected areas such as joint or disc spaces where the cartilage has deteriorated.

Osteoarthritis refers to a degenerative joint disease occurring chiefly in older persons, characterized by degeneration of the articular cartilage, hypertrophy of bone at the margins, and often limited inflammatory changes in synovial membrane. It is accompanied by pain and stiffness, particularly after prolonged activity. It is a form of arthritis where osteophytes are present in one or more joints.

Generalized osteoarthritis refers to a variant form of osteoarthritis that develops spontaneously and affects numerous joints with no readily identifiable cause. Its mains symptoms are pain and degeneration of joint cartilage, however, it shows a marked predilection for the fingers, knees, hips, feet and spine with considerable remodeling of bone tissue. It is a form of arthritis where osteophytes are present in one or more joints and there is also hand osteoarthritis as indicated by≧2 Heberden's nodes in at least 1 hand. Heberden's nodes refers to small hard nodules, formed usually at the distal interphalangeal articulations of the fingers produced by calcific spurs of the articular cartilage and associated with interphalangeal osteoarthritis. Rheumatoid arthritis (RA) is a chronic systemic inflammatory disease of undetermined etiology involving primarily the synovial membranes and articular structures of multiple joints. The disease is often progressive and results in pain, stiffness, and swelling of joints. In late stages deformity and ankylosis develop. The cause of RA is unknown. Factors associated with RA include the possibility of infectious triggers, genetic predisposition, and autoimmune response. The primary targets of inflammation are synovial membranes and articular structures. Other organs are affected as well. Inflammation, proliferation, and degeneration typify synovial membrane involvement. Joint deformities and disability result from the erosion and destruction of synovial membranes and articular surfaces.

A biological sample, as defined by the present invention, can be from human, dog, bovine, horse, guinea pig, sheep, pig, rabbit, mouse or rat. In addition, it can be selected from the group consisting of synovial fluid, serum, plasma, urine, broncheoalveolar lavage, medium extracts and cartilage extracts.

The invention is further illustrated by, though in no way limited to, the following examples.

### EXAMPLE 1

Subjects with knee osteoarthritis (OA), as defined by osteophyte presence in 1 or both knees, were divided into 2 groups, namely: (1) by the presence (knee OA+hand OA by≧2 Heberden's nodes in≧1 hand) or absence (knee OA without hand OA) of evidence of generalized OA. ELISA C2C and C1,2C assays and knee x-rays (semi-flexed with fluoro confirmation) were performed at baseline and 18 months. Progression was defined as a baseline-to-18-month increase in joint space narrowing grade. Progression was also examined as a K/L grade increase. Knees with the highest grade at baseline were excluded. Odds ratios for progression were estimated from logistic regression using generalized estimating equations to validly use data from both knees.

In the 209 subjects with knee OA, mean colratio was 4.40 (S,D. 11.00, range 0.17-94.03). In the 63 subjects with generalized OA, mean colratio in the progressors was 4.60 and in the non-progressors 4.08. In the 146 subjects without evidence of generalized OA, mean colratio was 6.71 and 3.64 in the progressors and non-progressors, respectively. Baseline colratio predicted baseline to 18 month progression in the group without generalized OA but not in the group with generalized OA. Each 20-unit increment in colratio was associated with a significant 1.46-fold increase in the odds of joint space progression, and a 1.78-fold increase in the odds of K/L progression. C1,C2 and C2C separately were not predictive of progression.

In summary, baseline serum colratio (C1,C2/C2C) predicted knee OA progression over the following 18 months, only in the subset without evidence of generalized OA. The ratio may be more predictive of progression than individual assay values since it may reflect increased secondary cleavage of type II collagen (with selective cleavage of the larger C2C epitope giving rise to the C1,2C epitope) based upon results from other studies.

### EXAMPLE 2

Subjects with early rheumatoid arthritis (usually involving multiple joints) were examined clinically at Visit 1 (inclusion at 0 month) at Visit 2 (at 18 months), and subsequently at Visit 3 (at 30 months). At each visit, sera were prepared for analysis by ELISA C2C and C1,2C assays. Knee, hand and foot x-rays were performed at each visit. Joint damage was recorded using the Sharpe/van der Heijde grading system based on analyses of x-rays for joint erosions, joint space narrowing, and total joint damage (total score). To date correlative analyses have been performed of the interrelationships between C2C and C1, 2C epitopes and their ratio at different visits with changes in joint erosions, joint space narrowing, and total joint scores between visits.

There was no detectable correlation for the assays (at Visit 1) with clinical score changes between Visit 1 (early disease presentation) and Visit 2. There were, however, significant correlations for the assays at Visit 2 with the clinical changes from Visit 2 to Visit 3. Thus, where n=number of patients, r is the correlation coefficient and p is the significance value (p=0.05 or less is significant), Spearman rank correlation analyses revealed that changes in hand erosions (n=20) (from Visits 2 to 3) correlated inversely with C2C assay at Visit 2 (r=−0.4554, p=0.0436), C1, 2C at Visit 2 correlated inversely with changes from Visit 2 to Visit 3 in joint space narrowing of the hand (n=20, r=−0.4523, p=0.0452) and of the foot (n=20, r=−0.4358, p=0.0547) and with total score change in all joints (n=20, r=−0.4564, p=0.0431). The ratio of C1, 2C to C2C at Visit 2 correlated inversely with the change in clinical score from Visit 2 to Visit 3 for foot erosions (n=20, r=−0.5660, p=0.0093) and for total score for the foot (n=20, r=−0.5700, p=0.0087).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

All of the publications and patent applications and patents cited in this specification are herein incorporated in their entirety by reference.

We claim:

1. A method of predicting the progression of osteoarthritis or other types of arthritis involving joint destruction, comprising determining a ratio of C1, 2C neoepitope to C2C neoepitope, wherein a higher result of said ratio is predictive of a greater progression of joint destruction of the arthritis.

2. The method according to claim 1, wherein a higher result of said ratio is predictive of a greater progression of osteoarthritis.

3. The method according to claim 1, wherein said osteoarthritis is knee osteoarthritis.

4. A method of predicting the progression of rheumatoid arthritis or other types of arthritis involving joint destruction, comprising determining a ratio of C1, 2C neoepitope to C2C neoepitope, wherein a lower result of said ratio ratio is predictive of a greater progression of rheumatoid arthritis.

5. The method according to claims 1 or 4, wherein said subject does not exhibit generalized osteoarthritis or exhibits rheumatoid or other inflammatory erosive arthritis.

6. A method of predicting the progression of arthritis involving joint destruction other than osteoarthritis, comprising determining a level of C2C neoepitope and/or C1, 2C neoepitope in a subject, wherein a lower result of said level is predictive of a greater progression of joint destruction of the arthritis.

7. The method according to claim 6, wherein a lower result in C2C neoepitope and/or C1, 2C neoepitope level is predictive of a greater progression of rheumatoid arthritis.

8. A method of monitoring osteoarthritis, rheumatoid arthritis, or other types of arthritis involving joint destruction, comprising determining a ratio of C1, 2C neoepitope to C2C neoepitope in a subject, wherein an increase or change in said ratio indicates an increase or change in rate of progression of joint destruction of the arthritis.

9. The method according to claim 8, wherein an increase in the ratio indicates a progression of osteoarthritis.

10. The method according to claim 8, wherein a decrease in the ratio indicates a progression of rheumatoid arthritis.

11. The method according to claim 1, wherein the subject does not exhibit generalized osteoarthritis or exhibits rheumatoid arthritis or other inflammatory erosive arthritis.

12. The method according to claim 1, wherein said osteoarthritis is knee osteoarthritis.

13. A method of monitoring arthritis involving joint destruction other than osteoarthritis, comprising determining a level of C2C neoepitope and/or C1, 2C neoepitope in a subject, wherein a change in said level indicates a change in rate of progression of joint destruction of the arthritis.

14. The method according to claim 13, wherein a decrease in C2C neoepitope and/or C1, 2C neoepitope level indicates an increase in rate of progression of rheumatoid arthritis.

15. A method of monitoring efficacy of a therapeutic treatment for osteoarthritis, rheumatoid arthritis or other types of arthritis involving joint destruction, comprising determining a ratio of C1,2C neoepitope to C2C neoepitope in a subject, wherein a decrease or change in the ratio indicates or relates to a decrease or change in the rate of progression of joint destruction of the arthritis.

16. The method according to claim 15, wherein the subject does not exhibit generalized osteoarthritis or exhibits rheumatoid arthritis or other types of arthritis involving joint destruction.

17. The method according to claim 15, wherein an increase in said ratio indicates a progression of osteoarthritis.

18. The method according to claim 15, wherein a decrease in said ratio indicates a progression of rheumatoid arthritis.

19. The method according to claim 15, wherein said osteoarthritis is knee osteoarthritis.

20. A method of monitoring efficacy of a therapeutic treatment for arthritis involving joint destruction other than osteoarthritis, comprising determining a level of C2C neoepitope and/or C1, 2C neoepitope in a subject, wherein a change in the level indicates or relates to a change in the rate of progression of joint destruction of the arthritis.

21. The method according to claim 20, wherein a decrease in C2C neoepitope and/or C1, 2C neoepitope level indicates an increase in rate of progression of rheumatoid arthritis.

22. A method of identifying an agent for treating osteoarthritis, rheumatoid arthritis or other types of arthritis involving joint destruction, comprising administering to a subject an agent to be tested, and determining a ratio of C1,2C neoepitope to C2C neoepitope in said subject, wherein a decrease or change in the ratio indicates or relates to a decrease or change in the rate of progression of joint destruction of the arthritis.

23. The method according to claim 22, wherein a decrease in said ratio indicates a decrease in the progression of osteoarthritis.

24. The method according to claim 22, wherein said osteoarthritis is knee osteoarthritis.

25. The method according to claim 22, wherein an increase in said ratio indicates a decrease in the progression of rheumatoid arthritis.

26. The method according to claim 22, wherein the subject does not exhibit generalized osteoarthritis, or exhibits rheumatoid arthritis or other types of arthritis involving joint destruction.

27. A method of identifying an agent for treating arthritis involving joint destruction other than osteoarthritis, comprising determining a level of C2C neoepitope and/or C1, 2C neoepitope in a subject, wherein a change in said level indicates a change in rate of progression of joint destruction of the arthritis.

28. The method according to claim 27, wherein a decrease in C1,2C neoepitope and/or C 2C neoepitope level indicates an increase in rate of progression of rheumatoid arthritis.

29. A pharmaceutical composition for osteoarthritis, rheumatoid arthritis or other types of arthritis involving joint destruction comprising an agent identified by the method of claims 22 or 27 in an amount effective to decrease or change the ratio of C1,2C neoepitope to C2C neoepitope in a subject or reduce or alter the amount of the increase or change in said ratio relative to an untreated subject.

30. The pharmaceutical composition according to claim 29, wherein an effective amount of said composition decreases said ratio, thereby slowing progression of osteoarthritis.

31. The pharmaceutical composition according to claim 29, wherein said osteoarthritis is knee osteoarthritis.

32. The pharmaceutical composition according to claim 29, wherein an effective amount of said composition increases said ratio, thereby slowing progression of rheumatoid arthritis.

33. The pharmaceutical composition according to claim 29, wherein said subject does not exhibit generalized osteoarthritis, or exhibits rheumatoid arthritis or other types of arthritis involving joint destruction.

34. A kit for for determining a ratio of C1,2C neoepitope to C 2C neoepitope in a biological sample, said kit comprising:
(a) a monoclonal antibody which binds to said C2C neoepitope having the following first peptide sequence
C-G-G-E-G-P-P(OH)-G-P-Q-G (COL2-¾C_{long mono} peptide);
(b) a polyclonal or monoclonal antibody which binds to said C1, 2C neoepitope having the following second peptide sequence
C-G-P-P(OH)-G-P-Q-G (COL2-¾Cₛₕₒᵣₜ peptide)
(c) two solid supports for binding each of said first and second peptides;
(d) a first labelled antibody conjugated to a first enzyme to measure the binding of said monoclonal antibody to said first peptide containing the C2C neoepitope; and
(e) a second labelled antibody conjugated to a second enzyme to measure the binding of said polyclonal or monoclonal antibody to said second peptide containing the C1, 2C neoepitope.

35. The kit according to claim 34, wherein said biological sample is selected from the group consisting of synovial fluid, serum, plasma, urine, broncheoalveolar lavage, medium extracts and cartilage extracts.

36. The kit according to claim 34, wherein said biological sample is from a human, dog, bovine, horse, guinea pig, sheep, pig, rabbit, mouse or rat.

37. The kit according to claim 34, wherein said first and second enzymes are the same or different.

38. The kit according to claim 34, wherein said first or second enzyme is alkaline phosphatase or hydrogen peroxidase.

39. The kit according to claim 34, wherein an increase or change in said ratio indicates an increase or change in rate of progression of joint destruction of osteoarthritis.

40. The kit according to claim 34, wherein said osteoarthritis is knee osteoarthritis.

41. The kit according to claim 34, wherein a decrease or change in said ratio indicates an increase or change in rate of progression of joint destruction of rheumatoid arthritis.

42. The kit according to claim 34, wherein said biological fluid from a subject does not exhibit generalized osteoarthritis or exhibits rheumatoid arthritis or other types of arthritis involving joint destruction.

43. The kit according to claim 34, wherein wherein a higher result of said ratio is predictive of greater progression of joint destruction of osteoarthritis.

44. The kit according to claim 34, wherein wherein a lower result of said ratio is predictive of greater progression of rheumatoid arthritis.

45. In a method of treating osteoarthritis, rheumatoid arthritis or other types of arthritis involving joint destruction, the improvement comprising determining a ratio of C1,2C neoepitope to C2C neoepitope in a subject being treated, wherein a decrease or change in said ratio correlates to a decrease or change in rate of progression of joint destruction of the arthritis.

46. In the method according to claim 45, wherein a decrease in said ratio indicates a decrease in the progression of osteoarthritis.

47. In the method according to claim 45, wherein said osteoarthritis is knee osteoarthritis.

48. In the method according to claim 45, wherein an increase in said ratio correlates to a decrease in the progression of rheumatoid arthritis.

49. In the method according to claim 45, wherein said subject does not exhibit generalized osteoarthritis, or exhibits rheumatoid arthritis or other types of arthritis involving joint destruction.

50. In a method of treating arthritis involving joint destruction other than osteoarthritis, the improvement comprising determining a level of C2C neoepitope and/or C1, 2C neoepitope in a subject, wherein a change in the level indicates a change in rate of progression of joint destruction of the arthritis.

51. In said method according to claim 50, wherein an increase in C2C neoepitope and/or C1, 2C neoepitope level indicates a decrease in rate of progression of rheumatoid arthritis.
